(19) United States
(12) Patent Application Publication
(10) Pub. No.: US 2014/0135392 A1
(43) Pub. Date: May 15, 2014

(54) METHODS FOR THE TREATMENT OF SIALORRHEA

(71) Applicant: NEURX PHARMACEUTICALS LLC, Salt Lake City, UT (US)

(72) Inventor: Dinesh C. Patel, Salt Lake City, UT (US)

(73) Assignee: NeuRx Pharmaceuticals LLC, Salt Lake City, UT (US)

(21) Appl. No.: 13/675,778

(22) Filed: Nov. 13, 2012

(51) Int. Cl. A61K 31/216

(52) U.S. Cl. CPC A61K 31/216
USPC 514/534

(57) ABSTRACT

The present invention provides safe and effective methods for the treatment of sialorrhea (excessive drooling) by administering an effective amount of N-desethyloxybutynin, or an optical R- or S-isomer thereof. N-desethyloxybutynin is a metabolite of oxybutynin and has higher affinity for receptors present in the parotid glands (the salivary glands in humans) than oxybutynin.

### FIELD OF INVENTION

The present invention relates to methods of treating sialorrhea (excessive drooling) by administering N-desethyloxybutynin to a subject in need of such treatment.

### BACKGROUND OF THE INVENTION

Sialorrhea, commonly known as excessive drooling, or hypersalivation, is the inability to control oral secretions resulting in excessive accumulation and involuntary loss of saliva from the mouth. In a normal healthy individual, there is a balance between the production of saliva in the mouth by the salivary glands and the swallowing reflex which eliminates pooling of saliva from the oropharynx. When this balance is perturbed by increased saliva production and/or decreased swallowing reflex, it leads to the pooling of saliva in the mouth and consequent involuntary loss.

Sialorrhea is one of the major non-motor complaints in patients suffering from various neurological impairments, including Parkinson's disease, cerebral palsy, Amyotropic Lateral Sclerosis, Huntington's disease, stroke and traumatic brain injury. Sialorrhea is also a commonly occurring side effect of antipsychotic medications.

Sialorrhea leads to a range of physical and psychosocial complications including perioral chapping, dehydration, odor and social embarrassment and isolation. Sialorrhea is often described by these patients as one of the most significant disabling social problems of their disease. Depending on its severity, drooling can result in medical disability, impaired speech or serious eating difficulties.

### SUMMARY OF THE INVENTION

In view of the foregoing, the inventor has recognized a need for a drug treatment for sialorrhea that will allow for the therapeutic effect of reducing salivary flow, but having fewer side effects. Such a drug for the treatment of sialorrhea therapy would preferably be selective to the salivary glands.

Accordingly, the present invention relates to a method of treating sialorrhea by administering an effective amount of N-desethyloxybutynin, pharmaceutically acceptable salts of N-desthyloxybutynin, isomers of N-desethyloxybutynin, pharmaceutically acceptable salts of isomers of N-desethyloxybutynin or mixtures thereof to a subject in need of the treatment.

Oxybutynin is a racemic mixture and has a chiral molecular center leading to the presence of (R)- and (S)-isomers. When metabolized, oxybutynin gives rise to metabolites such as N-desethyloxybutynin, which may also be present as (R)- and (S)-isomers or a combination thereof. The methods of the present invention specifically encompass administration of N-desethyloxybutynin as a free base, a pharmaceutically acceptable salt, isomers of the free base or salt and the like. For example, the present invention also encompasses the administration of each isomer of N-desethyloxybutynin individually or in combination for the treatment of sialorrhea.

The N-desethyloxybutynin of the present invention is administered as a suitable pharmaceutical dosage form or composition and may include pharmaceutically acceptable carriers and other ingredients as dictated by the particular needs of the dosage form. Such ingredients are well known to those skilled in the art. See for example, Gennaro, A. Remington: The Science and Practice of Pharmacy 19^{th} ed. (1995), which is incorporated by reference in its entirety. Unlike previous references to N-desethyloxybutynin, which involve this metabolite being formed in situ following administration of oxybutynin for treatment of urinary incontinence, the present invention is drawn to the direct administration of N-desethyloxybutynin for the treatment of sialorrhea. The prior art has, in fact, taught that the minimizing the formation of N-desethyloxybutynin is desirable.

Examples of suitable dosage forms for administration include oral, parenteral, buccal, transdermal, inhalant, implantable, vaginal or rectal type compositions. In one preferred aspect, the composition is an oral composition.

These and other embodiments of the invention, and their features and characteristics, will be described in further detail in the description and claims that follow.

### BRIEF DESCRIPTION OF THE DRAWINGS

FIG. 1 shows the chemical structure of oxybutynin.

FIG. 2 shows the chemical structure of N-desethyloxybutynin.

### DEFINITIONS

For convenience, before further description of the present invention, certain terms employed in the specification, examples and appended claims are collected here. These definitions should be read in the light of the remainder of the disclosure and as understood by a person skilled in the art. Unless defined otherwise, all technical and scientific terms used herein have the same meaning as would be understood by a person of ordinary skill in the art.

The singular forms “a”, “an” and “the” include plural references unless the context clearly dictates otherwise. Thus for example, reference to “an excipient” includes references to one or more of such excipients.

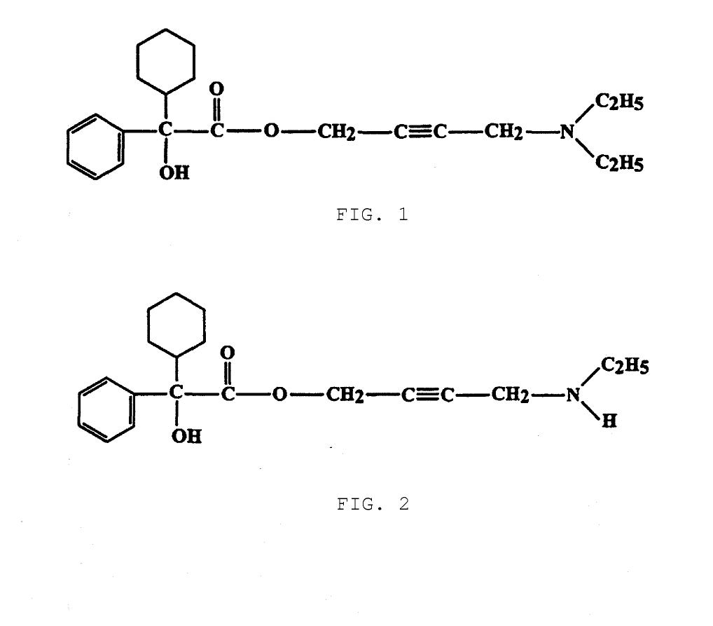

N-desethyloxybutynin is an active metabolite of oxybutynin, a drug commonly used for the treatment of urinary incontinence. The IUPAC name for N-desethyloxybutynin is 4-(ethylamino)but-2-ynyl2-cyclohexyl-2-hydroxy-2-phenylacetate. N-desethyloxybutynin is also referenced in the literature as 4-ethylamino-2-butynyl cyclohexyl-phenylgllycolate. The chemical structure of oxybutynin is shown in FIG. 1. The chemical structure of N-desethyloxybutynin is shown in FIG. 2. N-desethyloxybutynin also includes R- and S-isomers and all functional salts of N-desethyloxybutynin and its isomers. As noted above, the administration of N-desethyloxybutynin directly for the treatment of sialorrhea, rather than being formed as a consequence of oxybutynin administration in the treatment of urinary incontinence, is a novel feature which has not heretofore been taught.

As used herein, the term “about” means that dimensions, formulations, parameters, and other quantities and characteristics are not and need not be exact, but may be approximated and/or larger or smaller, as desired, reflecting tolerances, conversion factors, rounding off, measurement error and the like and other factors known to those of skill. Further, unless otherwise stated, the term “about” shall expressly include “exactly,” consistent with the discussion above regarding ranges and numerical data.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

“Administration” and “administering” refers to the manner in which a drug is presented to a subject. “Direct administration” may refer to administration of a specified active agent or drug per se, as compared to administration of an agent that metabolizes in-vivo to produce a specified active agent, such as an active metabolite (i.e. indirect administration). Such direct administration may in some regards provide more potent and targeted therapies with a lower incidence of adverse or undesirable side effects as compared to indirect administration. Further, direct administration of an active agent is most often a more potent therapy than indirect administration. As such, a smaller amount of active agent can be delivered in order to achieve a therapeutic effect.

Administration can be accomplished by various routes well known in the art such as oral, parenteral, buccal, transdermal, inhalation, implantation, vaginal or rectal. Thus, oral administration can be achieved by administering the drug as a solid e.g. pill, tablet, capsule, lozenge, suppository and the like which may be swallowed, chewed, or sucked Oral administration may also be by means of a liquid such as an oral solution, syrup or suspension of the drug. Oral administration includes immediate as well as controlled release formulations which encompass slow release, sustained release, extended release, prolonged release and delayed release. Parenteral administration can be achieved by injecting a drug composition intravenously, intra-arterially, intramuscularly, intrathecally or subcutaneously, etc. Parenteral administration includes immediate as well as controlled or sustained release formulations. Transdermal administration can be accomplished by applying, pasting, rolling, pouring, pressing, nibbling etc., of a transdermal composition onto a skin surface such as by means of a transdermal patch, creams, ointments etc. Transmucosal administration may be achieved by administering an the drug to a subject through a mucosal membrane. One form of transmucosal administration is buccal administration through the oral mucosal. Buccal administration may in some aspects also be considered as a form of oral administration and can be achieved by means of compositions that are designed to dissolve in the mouth, adhere to the gum or inside of the cheek, or be held sublingually, etc. Transmucosal administration may also be achieved in some aspects by insertion of the drug into the vaginal or anal cavities.

The term “pharmaceutically acceptable salts” used interchangeably with “salts”, is recognized in the art and refers to salts prepared from relatively non-toxic acids or bases including inorganic acids and bases and organic acids and bases.

The terms “pharmaceutically acceptable carrier” and “carrier, as used herein are well known to those skilled in the art and may be used interchangeably and refers to any inert and pharmaceutically or nutritionally acceptable material with which the bioactive agent may be combined to achieve a specific dosage formulation for delivery to a subject. As a general principle, carriers must not react with the bioactive agent in a manner which substantially degrades or otherwise adversely affects the bioactive agent. See for example, Gennaro, A. Remington: The Science and Practice of Pharmacy 19^{th} ed. (1995), which is incorporated by reference in its entirety. Specifically selected carriers often depend on the type of dosage form. For a transdermal patch, the carrier is typically a pressure sensitive adhesive into which the drug and other excipients are incorporated; the patch is then affixed to the skin to effect delivery of the drug. For a tablet dosage form, a powder carrier is formed by admixing the drug with excipients that can act as fillers, flow property modifiers, compressibility modifiers, control release agents, lubricants etc. that enable the powder to be compressed into a tablet. Other carriers conventionally known and used in the art are meant to be included in this definition unless specifically excluded.

As used herein, “excipient” and similar terms refers to substantially inert substances, which may be combined with an active agent and a carrier to achieve a specific dosage formulation for delivery to a subject, or to provide a dosage form with specific performance properties. For example, excipients may include binders, lubricants, etc., but specifically exclude active agents and carriers.

The term “subject” refers to a mammal that may benefit from the administration of N-desethyloxybutynin according to the method of this invention as expressed herein. Examples of subjects include humans as well as other warm-blooded animals such as horses, pigs, cattle, dogs, cats, rats or mice, etc. Preferably the subject will be a human.

The term “formulation” is used interchangeably with “composition”.

The terms “drug”, “active agent or ingredient” and “pharmaceutical” are also used interchangeably to refer to the pharmacologically active substance, i.e. N-desethyloxybutynin including isomers salts, mixtures as defined herein, present in the formulation or composition. These terms are well known in the pharmaceutical and medical arts.

As used herein, “effective amount” refers to an amount of N-desethyloxybutynin which, when included in a composition, is sufficient to achieve an intended compositional or physiological effect. Thus, a “therapeutically effective amount” refers to a non-toxic, but sufficient amount of N-desethyloxybutynin, to achieve therapeutic results in treating or preventing a sialorrhea. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of an effective amount is well within the ordinary skill in the art of pharmaceutical and nutritional sciences as well as medicine. Effective amounts of a N-desethyloxybutynin may be administered in a single dose or multiple doses.

The term “mean”, or “average” or similar terms when used in conjunction with the recitation of a number or numbers, means the sum of all the individual observations or items of a sample divided by the number of items in the sample.

If used, the phrase “area under the curve (AUC)” or “area under the plasma concentration-time curve” or similar terms are well known in the art.

The terms “comprise” and “comprising” are used in the inclusive, open sense, meaning that additional elements may be included.

The terms “consisting”, “consisting of” or “consisting essentially of” are used to limit the elements to those specified and those that do not materially affect the basic and novel characteristics of the material, i.e. composition or formulation or steps of administering the same.

### DETAILED DESCRIPTION OF THE INVENTION

Sialorrhea, commonly known as excessive drooling, is defined as the inability to control oral secretions resulting in excessive accumulation and involuntary loss of saliva from the mouth. In a normal healthy individual, there is a balance between the production of saliva in the mouth and the swallowing reflex which eliminates the saliva from the oropharynx. When this balance is perturbed either by increased saliva production and/or decreased swallowing reflex, it leads to drooling, the pooling of saliva in the mouth and consequent involuntary loss.

Sialorrhea is one of the major non-motor complaints in patients suffering from various neurological impairments, including Parkinson's disease (PD), cerebral palsy, Amyotropic Lateral Sclerosis (ALS), Huntington's disease, stroke and traumatic brain injury. Sialorrhea is also a commonly occurring side effect of antipsychotic medications, particularly clozapine.

Sialorrhea may affect up to one million patients with diverse neurological diseases. It affects a large proportion of PD patients, ranging up to 78% in advanced stages, with many PD patients considering drooling as their worst non-motor symptom. See for example, Kalf, J. G., J. Neurol. 2009; 256:1391-1396. Hypersalivation occurs in approximately one-third of schizophrenia patients treated with clozapine, a widely prescribed antipsychotic medication. See for example, Azorin, J-M., et al. Am. J. Psychiatry 2001; 158:1305-1313. Other large target populations include cerebral palsy patients and millions of survivors of stroke and severe traumatic brain injury.

CUVPOSA® (glycopyrrolate, an anticholinergic approved by the FDA on Jul. 28, 2010), marketed by Shionogi Pharma, is the only approved drug to “reduce chronic severe drooling in patients aged 3 to 16 years with neurologic conditions associated with problem drooling (e.g. cerebral palsy)”. It has a narrow orphan indication for the treatment of severe drooling in cerebral palsy patients and is available as 1 mg/5 ml oral solution. It has to be taken three times a day, one hour before or two hours after meals. The dose has to be carefully titrated from a low starting dose in increments of 0.02 mg/kg every 5-7 days. There are no other approved treatments currently known for sialorrhea in the United States.

The FDA approved treatment referenced above has significant disadvantages with respect to patient compliance and convenience as well as unwanted side effects. Therefore the present inventor recognizes a need for a drug therapy for sialorrhea that is efficacious, with minimal anticholinergic side effects and improved patient convenience and compliance.

Oxybutynin, an anticholinergic/antimuscarinic agent, is an approved drug for the treatment of urinary incontinence. Oxybutynin is available in oral as well as transdermal dosage forms and is marketed under such tradenames as Ditropan® (immediate release tablet), Ditropan® XL (extended release tablet), Oxytrol® (transdermal patch), and Gelnique® (transdermal gel).

Oxybutynin is a racemic mixture and has a chiral molecular center leading to the presence of (R)- and (S)-isomers. Particularly (R)-oxybutynin has been thought to be the more active of the two isomers, as indicated by animal pharmacological studies using isolated tissues. See for example, Kachur J F, Peterson J S, Carter J P, et al. J. Pharm. Exper. Ther. 1988; 247:867-872; see also Noronha-Blob L, Kachur J F. J. Pharm. Exper. Ther. 1990; 256: 560-567.

N-desethyloxybutynin is a metabolite of oxybutynin and is present as a racemic mixture or isolated as the (R)— or (S)—N-desethyloxybutynin isomer. The structures of oxybutynin (OXY) and N-desethyloxybutynin (DEO) are shown in FIGS. 1 and 2 respectively. For comparative activities of the anticholinergic/antimuscarinic and other activities of oxybutynin (OXY) and N-desethyloxybutynin (DEO) see U.S. Pat. No. 5,677,346; U.S. Pat. No. 6,432,446; U.S. Pat. No. 6,123,961; and U.S. Publication 2002/0002201 all of which are expressly incorporated herein in their entirety by reference.

When oxybutynin is administered orally it undergoes extensive first pass metabolism; the absolute bioavailability of oxybutynin is ˜6%. It is extensively metabolized first pass in the liver to N-desethyloxybutynin. Circulating plasma concentrations of N-desethyloxybutynin are 5-10 times higher than the oxybutynin plasma concentrations following oral administration of oxybutynin. This is true regardless of whether the oxybutynin is administered as an immediate release tablet or as an extended release tablet. When oxybutynin is administered transdermally, it bypasses first-pass metabolism by the liver, resulting in significantly lower plasma concentration of N-desethyloxybutynin than the oral route. N-Desethyloxybutynin, and the R- and S-isomers thereof, are thought to be the active metabolites of oxybutynin that are responsible for much of the adverse effects, such as dry mouth, associated with the use of oxybutynin. However, this is the result of administering oxybutynin to a subject and not the direct administration of N-desethyloxybutynin or an isomer. See, Reitz et al. “The preparation and human muscarinic receptor profiling of oxybutynin and N-desethyloxybutynin enantiomers”, Med. Chem. 3 (6)” 343-5, (2007). Following oxybutynin administration N-Desethyloxybutynin plasma levels may reach as much as ten times that of the parent drug after administration of the immediate-release oral formulation. See Zobrist et al. “Pharmaacokinetics of the R- and S-Enantiomers of Oxybutynin and N-Desethyloxybutynin Following Oral and Transdermal Administration of the Racemate in Healthy Volunteers”. Pharmaceutical Research 18:1029-1034, (2001). Alternative dosage forms have been developed in an effort to reduce blood levels of N-desethyloxybutynin and allow for a more steady concentration of oxybutynin to be achieved than is possible with the immediate release form. See U.S. Pat. No. 6,262,115; U.S. Pat. No. 5,912,268; U.S. Pat. No. 5,840,754 and U.S. Pat. No. 5,674,895 all of which are expressly incorporated herein in their entirety.

While the above focuses on the negative aspects that might be attributed to N-Desethyloxybutinyn and its isomers, is the result of administering oxybutynin to a subject suffering from urinary incontinence and having it internally metabolized into N-desethyloxybutynin and other metabolites. The isolation and direct administration of this metabolite, without having first administered oxybutynin, and finding positive pharmacological uses for in non-urinary incontinence related conditions such as sialorrhea has heretofor not been recognized.

N-desethyloxybutynin is not an approved drug and no generic or trade name is known for N-desethyloxybutynin, its isomers or any of its salts. Whatever human or animal exposure of N-desethyloxybutynin exists in the prior art is a consequence of oxybutynin administration. Moreover, the mention of N-desethyloxybutynin in the prior art is generally used in a negative sense as a cause or contributor of unwanted side effects. Furthermore, any pharmacologic activity of N-desethyloxybutynin is usually associated with treatment of urologic conditions.

N-desethyloxybutynin will be referred to herein either by the name N-desethyloxybutynin or simply desethyloxybutynin or DEO when referring to it as a racemic mixture. Reference to a specific optical isomer will be referenced as R-desethyloxybutynin or R-DEO or S-desethyloxybutynin or S-DEO. Pharmaceutically acceptable salts of DEO, R-DEO and S-DEO will automatically be included unless specifically referred to otherwise, such as a free base of DEO.

It has now been unexpectedly determined that N-desethyloxybutynin and its R- and S-isomers, when properly administered, can be effective in the treatment of sialorrhea while having fewer side effects than other agents that have been proposed or used in the treatment of this condition.

Moreover, the selectivity of N-desethyloxybutynin for the parotid glands (glands behind or beside the ear, the salivary glands in humans) also shows that it is potentially safer than other compositions, such as other anticholinergics used alone or in combination with other agents for the treatment of sialorrhea. There is no teaching or suggestion in the literature or prior art that N-desethyloxybutynin can be used to treat sialorrhea and ameliorate the side effects commonly found when treating this excessive drooling condition.

The overall process for preparing DEO involves: (a) the preparation of the side chain 4-ethylamino-2-butynyl chloride from dichlorobutyne (b) by standard esterification technique, reacting cyclohexylphenyl glycolic acid with 4-ethylamino-2-butynyl chloride to produce 4-ethylamino-2-butynyl cyclohexylphenyl-glycolate (DEO).

An alternative process for preparing DEO involves the preparation of a hydroxylated side chain instead of the above mentioned halogenated side chain.

A process for preparing R-DEO is described in U.S. Pat. No. 6,123,961 and a process for preparing S-DEO is described in U.S. Pat. No. 5,532,278, the disclosures of which are hereby incorporated by reference in their entirety.

The magnitude of a prophylactic or therapeutic dose of the DEO compounds of this invention in the acute or chronic management of sialorrhea will vary with the severity and nature of the condition to be treated and the route of administration. The dose and the frequency of the dosing will also vary according to the age, body weight and response of the individual patient. In general, the total daily dose range for the compound of this invention for the conditions described herein is from about 1 mg to about 100 mg in single or divided doses, preferably in a single dose. In managing the patient, the therapy should be initiated at a lower dose, perhaps at about 5 mg to about 10 mg, and may be increased up to about 30-100 mg depending on the patient's global response. For immediate release dosages a oral dosage of about 1-30 mg administered once or multiple times a day is thought adequate. For sustained release dosages an oral dosage of about 2-100 mg may be administered once or twice a day. It is further recommended that patients over 65 years and those with impaired renal or hepatic function initially receive low doses and that they be titrated based on individual response(s) and plasma drug level(s). It may be necessary to use dosages outside these ranges, as will be apparent to those skilled in the art. Further, it is noted that the clinician or treating physician will know how and when to interrupt, adjust, or terminate therapy in conjunction with individual patient response. The terms “a therapeutically effective amount” and “an amount sufficient to treat sialorrhea but insufficient to cause adverse effects” are encompassed by the above-described dosage amounts and dose frequency/schedule.

Any suitable route of administration may be employed for providing the patient with an effective dosage of the compounds of this invention. For example, oral, sublingual, rectal, parental (subcutaneous, intramuscular, intravenous), intraocular, transdermal, aerosol and like forms of administration may be employed. Dosage forms include tablets, controlled-release tablets, troches, dispersions, suspensions, solutions, syrups, capsules, microencapsulated systems, sprays, transdermal delivery systems, and the like.

The pharmaceutical compositions of the present invention comprise N-desethyloxybutynin, its isomers, salts and combinations thereof as defined above as the active ingredient, and may also contain a pharmaceutically acceptable carrier, and optionally, other therapeutic ingredients.

The terms “pharmaceutically acceptable salts” or “a pharmaceutically acceptable salt thereof” refer to salts prepared from pharmaceutically acceptable non-toxic acids. Suitable pharmaceutically acceptable acid addition salts for the compound of the present invention include acetic, benzenesulfonic (besylate), benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pathothenic, phosphoric, succinic, sulfuric, tartaric, p-toluenesulfonic, and the like. The hydrochloride salt is particularly preferred for oral dosage forms.

The compositions of the present invention include suspensions, solutions, elixirs, powders or solid dosage forms (tablets and capsules). Carriers such as starches, sugars, and microcrystalline cellulose, diluents, granulating agents, lubricants, binders, disintegrating agents, and the like are suitable in the case of oral solid preparations (such as powders, capsules, and tablets). Oral solid preparations are preferred over the oral liquid preparations, except for administration in pediatric population where liquid dosage forms may be preferred.

Because of their ease of administration, tablets and capsules represent one of the more advantageous oral dosage unit forms, in which case solid pharmaceutical carriers are employed. If desired, tablets may be coated by standard aqueous or nonaqueous techniques. Since the compound of the invention has a relatively short duration of action in the body, it may be advantageous to administer the drug in a controlled-released or slow-release formulation, thereby decreasing the frequency of drug administration to the patient. The compounds of the present invention may also be administered by controlled release means and delivery devices such as those described in U.S. Pat. Nos. 3,845,770; 3,916,899; 3,536,809; 3,598,123; and 4,008,719, and PCT application WO92/20377, the disclosures of which are hereby incorporated by reference in their entirety. Various forms of controlled release or slow release transdermal administration forms and devices known in the art can also be used to improve the convenience of dosage for the patient and are hereby incorporated by reference.

As previously noted, pharmaceutical compositions of the present invention suitable for oral administration may be presented as discrete unit dosage forms such as capsules, cachets, or tablets, each containing a predetermined amount of the active ingredient, as a powder or granules, or as a solution or a suspension in an aqueous liquid, a non-aqueous liquid, an oil-in-water emulsion, or a water-in-oil liquid emulsion. Such compositions may be prepared by any of the methods of pharmacy, but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation, just as is known for the racemic mixture.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made by molding, in a suitable machine, a mixture of the powdered compound moistened with an inert liquid diluent. All of the foregoing techniques are well known to persons of skill in the pharmaceutical art. Each tablet may contain from about 1 mg to about 100 mg of the active ingredient.

The following provides examples of N-desethyloxybutynin compositions in accordance with the present invention. They are meant to be exemplary only and are not a restriction to the invention which is limited only by the following claims and functional equivalents thereof.

### Example 1

### Preparation of an Immediate Release N-Desethyloxybutynin HCl Tablet

After being sieved through #60 mesh screen 10 g of N-desethyloxybutynin HCL is transferred into a clean container. To this is added 1 g of silicon dioxide powder and mixed thoroughly to form a uniform blend Anhydrous lactose, 150 g, is sieved through a #60 mesh screen and transferred into a separate container. The N-desethyloxybutynin HCL-silicone blend and the lactose are then mixed thoroughly to obtain a uniform blend. To this uniform blend is added 1.5 g of magnesium stearate, which has also been sieved through a #60 mesh, which is then mixed thoroughly to again form a final uniform blend of active agent, silicon dioxide, lactose and magnesium stearate. Using a tablet press 162.5 mg portions of the final blend are pressed into tablets of the desired weight and shape each containing 10 mg of N-desethyloxybutynin HCl.

### Example 2

### Preparation of a Controlled Release N-Desethyloxybutynin HCl Tablet

After being sieved through a #60 mesh screen 30 g of N-desethyloxybutynin HCL is transferred into a clean container. To this is added 3 g of silicon dioxide powder and mixed thoroughly to form a uniform blend Anhydrous lactose, 100 g, is sieved through a #60 mesh screen and transferred into a separate container. The active agent, silicon dioxide and anhydrous lactose are blended thoroughly and to this blend is added 450 g of Methocel K4M that has been sieved through a #60 mesh screen. This blend is mixed thoroughly and to this is added 5 g of Magnesium stearate which has also been sieved through a #60 mesh screen. This powder blend is mixed until a uniform powder blend is obtained.

Using a tablet press this blend of 588 g uniformly blended powder is pressed into tablets using appropriate tooling to from tablets of the desired weight and shape each tablet containing 30 mg of N-desethyloxybutynin in a controlled release form.

### Example 3

### Preparation of a Controlled Release N-Desethyloxybutynin HCl Tablet

After being sieved through a #60 mesh screen 15 g of N-desethyloxybutynin HCL is transferred into a clean container. To this is added 1.5 g of silicon dioxide powder and mixed thoroughly to form a uniform blend Anhydrous lactose, 60 g, is sieved through a #60 mesh screen and transferred into a separate container. The active agent, silicon dioxide and anhydrous lactose are blended thoroughly and to this blend is added 150 g of Methocel K4M that has been sieved through a #60 mesh screen. This blend is mixed thoroughly and to this is added 1.5 g of Magnesium stearate which has also been sieved through a #60 mesh screen. This powder blend is mixed until a uniform powder blend is obtained.

Using a tablet press this blend of 228 g of uniformly blended powder is pressed into tablets using appropriate tooling to from tablets of the desired weight and shape each tablet containing 15 mg of N-desethyloxybutynin in a controlled release form.

### Example 4

### Preparation of a Controlled Release N-Desethyloxybutynin Transdermal Patch

The solids content of a pressure sensitive adhesive (PSA) (a solution of an acrylic adhesive polymer in organic solvents) is determined by weighing a small amount of adhesive solution in a pre-weighed aluminum dish. The solvent is then evaporated by overnight drying in a convection oven maintained at 70° C. and the percent solids adhesive content is determined from the ratio of the final dried weight to the initial solution weight.

To prepare a drug-containing adhesive film, 18 grams of the adhesive solution is weighed into a glass bottle. Assuming a percent solids adhesive content of 50%, this results in 9 grams of solid adhesive polymer. To this solution, 1 gram of N-desethyloxybutynin (free base) is added to yield a final desired dried film composition (% w/w) of acrylic adhesive/N-desethyloxybutynin 90/10. The glass bottle is tightly capped, sealed with parafilm, and rotated until the ingredients completely dissolve and the solution is visually clear.

Film casting of the adhesive-drug formulation is performed by dispensing approximately 10 ml of the adhesive/drug solution onto a polyester liner with a release coating and casting the solution as a thin film at a dry film coating weight of 6 mg/cm². The cast is dried in a convection oven at 70° C. for 15 minutes to yield the target dry film with a coating weight of 6 mg/cm². An occlusive polyethylene backing film is laminated onto the adhesive film to form a laminate. The laminate is then die-cut into 50 cm² patch and stored in sealed pouches. Each patch contains 30 mg of N-desethyloxybutynin.

### Example 5

### Administration of an Immediate Release N-Desethyloxybutynin HCl Tablet

The tablet of Example 1 containing 10 mg of N-desethyloxybutynin is orally administered three times a day to a patient suffering from sialorrhea and results in a 33% reduction in drooling.

### Example 6

### Administration of a Controlled Release N-Desethyloxybutynin HCl Tablet

The tablet of Example 2 containing 30 mg of N-desethyloxybutynin in controlled release form is orally administered once a day to a silaorrhea patient and results in 50% reduction in drooling within about 3 days of administration.

### Example 7

### Administration of a Controlled Release N-Desethyloxybutynin HCl Tablet

The tablet of Example 3 containing 15 mg of N-desethyloxybutynin in controlled release form is orally administered twice a day to a silaorrhea patient and results in 50% reduction in drooling within about 3 days of initial dosing.

### Example 8

### Administration of a Controlled Release N-Desethyloxybutynin Transdermal Patch

The transdermal patch of Example 4 is applied once daily to the upper arm of an individual suffering from sialorrhea resulting in 20% reduction in drooling within about 7 days of patch application.

The dosage form and the specific N-desethyloxybutynin form, i.e. salt, free base, R- and S-isomers or mixtures or isomers as specified above may be formulated and utilized in various dosages and forms, i.e. oral, buccal, transdermal, sublingual, injectable, and the like as would be obvious to one having ordinary skill in the art. It is to be understood that the above referenced compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements.

1. A method of treating sialorrhea which comprises directly administering to a subject having a need thereof a therapeutically effective amount of the active ingredient N-desethyloxybutynin, wherein the active ingredient comprises N-desethyloxybutynin; an isomer of N-desethyloxybutynin; a pharmaceutically acceptable salt of N-desethyloxybutynin or an isomer thereof, or a combination of any of the above.

2. The method of claim 1 where the N-desethyloxybutynin comprises (R)—N-desethyloxybutynin or a pharmaceutically acceptable salt thereof or (S)—N-desethyloxybutynin or a pharmaceutically acceptable salt thereof, or a combination thereof.

3. The method of claim 1 wherein the N-desethyloxybutynin is administered via oral, parenteral, buccal, transdermal, inhalation, implantation, vaginal or rectal route of administration.

4. The method of claim 3 wherein the N-desethyloxybutynin is administered via the oral route.

5. The method of claim 4 wherein the N-desethyloxybutynin is administered as an immediate release dosage form.

6. The method of claim 4 where in the N-desethyloxybutynin is administered as a controlled release dosage form.

7. The method of claim 5 where in the dosage form contains 1-30 mg of N-desethyloxybutynin.

8. The method of claim 6 where in the dosage form contains 2-100 mg of N-desethyloxybutynin.

9. The method of claim 1 wherein the N-desethyloxybutynin is administered via the buccal route.

10. A dosage form for administering N-desmethyloxybutynin to a patient comprising N-desmethyloxybutynin; an isomer of N-desmethyloxybutynin; a pharmaceutically acceptable salt of N-desmethyloxybutynin or an isomer thereof, or a combination of any of the above combined with a pharmaceutically acceptable carrier wherein the dosage form is a pill, tablet, capsule, lozenge, suppository, oral liquid, injectable liquid; or transdermal patch, cream or ointment.

11. A dosage form according to claim 10 wherein the N-desethyloxybutynin comprises (R)—N-desethyloxybutynin or a pharmaceutically acceptable salt thereof or (S)—N-desethyloxybutynin or a pharmaceutically acceptable salt thereof, or a combination thereof.

12. A dosage form according to claim 10 wherein the N-desethyloxybutynin is in a form capable of being administered via oral, parenteral, buccal, transdermal, inhalation, implantation, vaginal or rectal routes of administration.

13. A dosage form according to claim 10 wherein the N-desethyloxybutynin is formulated as a tablet for oral administration.

14. A dosage form according to claim 13 wherein the tablet is formulated for immediate release of N-desethyloxybutynin.

15. A dosage form according to claim 13 wherein the tablet is formulated for controlled release of N-desethyloxybutynin.

16. A dosage form according to claim 10 wherein the N-desethyloxybutynin is formulated as a supporitory for insertion into a body cavity.

17. A dosage form according to claim 10 wherein the N-desethyloxybutynin is formulated as a liquid for oral administration.

18. A dosage form according to claim 10 wherein the N-desethyloxybutynin is formulated as a liquid for parenteral administration.

19. A dosage form according to claim 10 wherein the N-desethyloxybutynin is formulated for transdermal administration.
